(19) United States
(12) Patent Application Publication
(10) Pub. No.: US 2022/0241459 A1
(43) Pub. Date: Aug. 4, 2022

(54) AN IMPLANT COMPRISING A COLLAGEN MEMBRANE

(71) Applicant: DATUM BIOTECH LTD., Lod (IL)

(72) Inventors: Ran KAPRI, Mazkeret Batya (IL); Thomas BAYER, Tel Aviv (IL); Arie GOLDLUST, Nes Ziona (IL)

(73) Assignee: DATUM BIOTECH LTD., Lod (IL)

(21) Appl. No.: 17/620,708

(22) PCT Filed: Jun. 18, 2020
(86) PCT No.: PCT/IL2020/050686

§ 371 Date: Dec. 19, 2021

(30) Foreign Application Priority Data
Jun. 20, 2019 (IL) PCT/IL2019/050690

(51) Int. Cl. A61L 27/24; A61L 27/06; A61L 27/16; A61L 27/54; A61L 27/58

(52) U.S. Cl. CPC A61L 27/24; A61L 27/06; A61L 27/16; A61L 27/54; A61L 2430/12; A61L 2300/404; A61L 2300/41; A61L 2300/412; A61L 2430/02; A61L 27/58

(57) ABSTRACT

The invention is directed to an implantable structure comprising at least one biocompatible backbone scaffold and at least one biocompatible and biodegradable collagen membrane deposited thereon, including methods of its preparation and uses thereof in dental or orthopedic bone regeneration, dura repairs, hernia repairs and similar procedures requiring structural implants.

### FIELD OF THE INVENTION

The invention is directed to an implantable structure comprising at least one biocompatible backbone scaffold and at least one biocompatible and biodegradable collagen membrane deposited thereon, including methods of its preparation and uses thereof in dental or orthopedic bone regeneration, dura repairs, hernia repairs and similar procedures requiring structural implants.

### BACKGROUND OF THE INVENTION

Many medical procedures require the implantation of various types of scaffolds or membranes into the body. Such scaffolds may be used, e.g., to prevent adhesion, to support certain organs or ligaments and/or to provide means for regenerating various types of tissue, such as cartilage, ligaments and bones. Every type of scaffold is required to remain in the body for a certain length of time. When repairing hernias, for example, the scaffold is required to remain permanently in the body, during which time it provides mechanical support to the stomach wall and prevents internal organs for being misplaced. Such a permanent implanted scaffold must be biocompatible and is further required to both provide support over a length of time and to be designed such that no infections and the like are caused in its vicinity. Other types of scaffolds, such as those used for guided tissue and bone regeneration, are required to remain in the body for shorter lengths of time, at least until the tissue/bone regeneration has been initiated, and possibly, until the tissue/bone regeneration has been completed. Although such scaffolds are also required to prevent infections in their vicinity; unlike permanently implanted scaffolds, they are not intended to be permanent and accordingly, may be designed to be both biocompatible and biodegradable.

One type of known biocompatible and biodegradable material is collagen. The collagen protein constitutes approximately 30% of the proteins in a living body and functions as a support for bone and cell adherence. Accordingly, collagen is known to be a useful biomaterial, used for example in cell culture substrates, as well as a scaffold material for regenerative medicine, including tissue engineering of cartilage, bone, ligaments, corneal stroma and skin. Collagen is used also as an implantation material, for example as a wound dressing material, bone grafting material, hemostatic material, or anti-adhesive material.

Although collagen is biocompatible and therefore, would not be rejected by the body and could prevent infections and its vicinity, implants prepared from collagen generally do not provide the required mechanical support and further, cannot be used when the implant is intended to be permanent, since the collagen implant tends to biodegrade. Further, since collagen membranes are generally prepared from processed tissue, the possibilities of combining the collagen with other materials, which could, theoretically, provide mechanical force to the collagen membrane, is limited.

Some types of permanent implants are known in the field, such as implants prepared from titanium, Teflon®, various types of polymers and the like. However, even though they are highly biocompatible, in many instances, the use of synthetic implants carries the risk of infections and biofilm formation in their vicinity, requiring their surgical removal and replacement. Accordingly, permanent implants that are both highly biocompatible and further, prevent infections in their vicinity are desired.

### SUMMARY OF THE INVENTION

The present invention provides an implantable structure comprising at least one biocompatible backbone scaffold and at least one biocompatible and biodegradable collagen membrane deposited on at least a part of said backbone surface.

According to some embodiments, this invention provides an implantable structure comprising at least one biocompatible backbone scaffold and at least one biocompatible and biodegradable collagen membrane deposited on at least a part of said backbone surface; wherein the biodegradation rate of the membrane is controllable. In another embodiment, the degradation rate of the collagen membrane is controllable based on the densities and/or crosslinking levels of the collagen membrane.

According to some embodiments, said backbone scaffold is formed from at least one of a metal, a polymeric agent and any combinations thereof. According to some embodiments, said backbone scaffold is formed of at least one of titanium, nitinol, polytetrafluoroethylene (PTFE, Teflon®), stainless steel, polypropylene, polystyrene, polyester, silicon, or any combination thereof. According to some embodiments, said backbone scaffold is in the form of a woven or non-woven mesh, wires, rods, or any combination thereof.

According to some embodiments, at least one biocompatible and biodegradable collagen membrane comprises one or more regions, each having a different degradation rate. According to some embodiments, at least one biocompatible and biodegradable collagen membrane further comprises at least one pharmaceutically active agent. According to some embodiments, said at least one pharmaceutically active agent is selected from antimicrobial agents, anti-inflammatory agents, factors having tissue regeneration induction properties and any combination thereof. According to some embodiments, said at least one biocompatible and biodegradable collagen membrane comprises crosslinked collagen. According to some embodiments, at least one biocompatible and biodegradable collagen membrane further comprises a space maintainer.

The invention further provides method of preparing an implantable structure as defined herein above and below the method comprising: providing at least one biocompatible backbone; immersing at least a part of said biocompatible backbone scaffold in a solution comprising collagen; fibrillating said collagen; and crosslinking said collagen thereby providing crosslinked collagen membrane deposited on the surface of said backbone. In another embodiment, the fibrillated collagen is optionally compressed by applying pressure (e.g. 1 kg) onto said collagen, thereby forming a collagen with high density. In another embodiment, the collagen is compressed before or after the crosslinking step.

According to some embodiments, the shape of said at least one biocompatible and biodegradable collagen membrane is defined according to the shape of the backbone scaffold it is formed thereupon. According to some embodiments, the solution comprises at least one fibrillation agent. According to some embodiments, said at least one fibrillation agent is selected from sodium phosphate or sodium hydroxide.

According to some embodiments, said at least one biocompatible and biodegradable collagen membrane is crosslinked by at least one of an enzymatic mediated process, heat, UV radiation, chemical crosslinking agent comprising a reducing sugar or a reducing sugar derivative, or any combination thereof. According to some embodiments, the reducing sugar or reducing sugar derivative includes an aldehyde or ketone mono sugar or mono sugar derivative wherein the α-carbon is in an aldehyde or ketone state in an aqueous solution.

According to some embodiments, said at least one crosslinking agent includes glycerose, threose, erythrose, lyxose, xylose, arabinose, ribose, allose, altrose, glucose, mannose, gulose, idose, galactose, talose, or any other diose, triose, tetrose, pentose, hexose, septose, octose, nanose or decose, or any combination thereof.

According to some embodiments, said at least one biocompatible and biodegradable collagen membrane comprises one or more regions, wherein each region is crosslinked to a different degree of crosslinking.

According to some embodiments, the method further comprises washing said at least one biocompatible and biodegradable collagen membrane to remove residual reactants. According to some embodiments, the method further comprises dehydrating the collagen membrane.

According to some embodiments, said implantable structure of the invention is used as an implanted device for dental or orthopedic bone regeneration, hernia repair, or dura repair. According to some embodiments, said implantable structure of the invention is used in conjunction with at least one space-maintainer.

The invention further provides a kit comprising an implantable structure as defined herein above and below.

In another aspect the invention provides a kit comprising at least one biocompatible backbone scaffold, at least one solution of collagen, and instructions for use thereof. According to some embodiments, said kit further comprises at least one fibrillation agent. According to some embodiments, said kit further comprises at least one crosslinker.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features and advantages thereof, may best be understood by reference to the following detailed description when read with the accompanied drawings. Embodiments of the invention are illustrated by way of example and not limitation in the figures of the accompanying drawings, in which like reference numerals indicate corresponding, analogous or similar elements, and in which:

FIG. 1 presents an implantable structure of the invention, wherein the backbone scaffold is a metal mesh.

FIG. 2 presents an implantable structure of the invention, wherein the backbone scaffold is a polymeric mesh.

FIG. 3 presents an implantable structure of the invention, wherein the backbone scaffold is a titanium mesh.

FIG. 4 presents an example of a three-dimensional implantable structure of an embodiment of the invention.

### DETAILED DESCRIPTION OF EMBODIMENTS OF THE INVENTION

The invention is directed to an implantable structure comprising at least one biocompatible backbone scaffold and at least one biocompatible and biodegradable collagen membrane deposited on at least a part of said backbone outer surface. Particularly, both the collagen membrane and the backbone scaffold are biocompatible, while only the collagen membrane is biodegradable. Accordingly, the collagen membrane mainly provides the implantable structure with the required biological properties, such as biocompatibility, biodegradation, and infection prevention, while the backbone scaffold mainly provides the implantable structure with the required physical and structural properties, including mechanical strength over a length of time.

In some embodiments, this invention provides an implantable structure comprising at least one biocompatible backbone scaffold and at least one biocompatible and biodegradable collagen membrane deposited on at least a part of said backbone surface; wherein the biodegradation rate of the membrane is controllable. In another embodiment, the degradation rate of the collagen membrane is controllable based on the densities and/or crosslinking levels of the collagen membrane.

According to some embodiments, it is herein defined that “collagen” within the collagen membrane of the invention is a pure, non-modified collagen. The collagen can be fibrillated and/or crosslinked (thus, fibrillating/crosslinking agents might be found within the collagen). Composites of collagen with e.g. other polymers, hydroxyapatite, carbon nanotubes, graphene and the like are excluded from this definition. Covalently modified collagen, especially (but no limited only to) conjugates of collagen with polymers are excluded as well.

When referring to an “implantable structure” (used interchangeably with the term “implant”, “article”, “structure”, “device” throughout) it should be understood to encompass a medical device manufactured to regenerate soft or hard tissues or organs, support a damaged soft or hard tissues or organs, or enhance an existing soft or hard tissues or organs. Said implantable structure of the invention comprises at least one biocompatible backbone scaffold that provides the general structural three-dimensional form of the implant which its surface is covered/coated with at least one collagen membrane. In some embodiments, only a part of the backbone is covered/coated with at least one membrane. In other embodiments, all of the backbone is covered/coated with at least one membrane (in some embodiments this also includes voids and internal spaces or pores within said backbone scaffold).

When referring to the term “deposited” relating to the deposition of at least one collagen membrane on at least a part of said backbone surface, it should be understood to encompass any possible form of deposition including coating, covering, dipping, forming of said membrane, putting, placing, and any combinations thereof, in any form chemical or mechanical or a combination thereof.

According to some embodiments, the tissue surrounding the implantable structure upon its implantation is regenerated, at least partially, over time, such that it replaces the collagen membrane. Thus, the collagen membrane may provide the necessary structure for the surrounding tissue to grow into. This may be essential, e.g., when the backbone scaffold provides support, however cannot provide the volume that the surrounding tissue, e.g., bone, is intended to fill. Thus, as the collagen membrane degrades, the surrounding tissue may take its place, essentially being built around the internal scaffold.

According to some embodiments, the collagen membrane in the implantable structure is prepared such that it is highly osteo-promotive. It is noted that the term “highly osteo-promotive” is meant to cover a collagen membrane that particularly encourages the growth of bone cells when there are bone cells in the vicinity thereof, even if other types of cells are also present; however, if there are no bone cells present, other types of tissue, not bone cells, are regenerated, taking the place of the biodegrading collagen. For example, if the implantable structure of the invention is implanted in the mouth between bone material and the gums, over time, as the collagen membrane biodegrades, bone cells will replace the collagen membrane, even on the side of the membrane that is adjacent to the gum tissue. Further, for example, if the implantable structure according to the invention is implanted in order to repair the skull, and is therefore placed between the skull and the skin covering it, the biodegraded collagen membrane will be replaced by bone cells, even on the side of the membrane adjacent to the skin. Nonetheless, if the implantable structure according to the invention is implanted where only soft tissue is present, e.g., when repairing a hernia, the collagen membrane will be replaced over time with the surrounding soft tissue.

According to some embodiments, the backbone scaffold is completely internal, i.e., it is completely covered by the collagen membrane of the structure of the invention. Accordingly, infections, prone to occur in the vicinity of the implants, especially close to the time of implantation, may be prevented, since the only material exposed to the body is the collagen membrane, which is highly biocompatible. The collagen membrane may also enable a slower exposure of the backbone scaffold, thereby reducing the tissue response to the implanted foreign structure. Rejection of the implant may also be prevented due to the high biocompatibility of the collagen membrane.

According to some embodiments, the collagen membrane is biodegradable and accordingly, the implantable structure of the invention is partially biodegradable. Once at least partially biodegraded, the backbone scaffold is exposed; however, since the implantable structure of the invention has already been in place for a period of time and the tissue has healed, the risk of infections is low. According to some embodiments, the collagen membrane is biodegraded within about 3-24 months. According to some embodiments, the collagen membrane is biodegraded within about 4-8 months. According to some embodiments, the collagen membrane is biodegraded within about 5-7 months. According to some embodiments, the collagen membrane is biodegraded within about 3-10 months. According to some embodiments, the collagen membrane is biodegraded within about 10-17 months. According to some embodiments, the collagen membrane is biodegraded within about 17-24 months. According to some embodiments, the collagen membrane is biodegraded within about six months. It is noted that the collagen membrane is considered biodegraded when more than about 70, 80, 90 or 95% is degraded.

According to some embodiments, the backbone scaffold is prepared from at least one of titanium, nitinol, stainless steel, any type of polymer, such as polytetrafluoroethylene (PTFE, Teflon®), polypropylene, polyester, polystyrene, silicon or any combination thereof.

According to some embodiments, the backbone scaffold is in any appropriate form, such as in the form of a perforated surface, a woven or non-woven mesh, wires, rods, and the like or any combination thereof. The thickness of the backbone scaffolds, or of any part thereof, may be in the range of about 0.05-1.0 mm. The thickness of the backbone scaffolds, or of any part thereof, may be in the range of about 0.2-2.0 mm. The thickness of the backbone scaffolds, or of any part thereof, may be in the range of about 1.0-3.0 mm. The thickness of the backbone scaffolds, or of any part thereof, may be in the range of about 3.0-5.0 mm. The thickness of the backbone scaffolds, or of any part thereof, may be in the range of about 5.0-7.0 mm. The shape of the backbone scaffold may be designed according to the required shape of the implantable structure of the invention including the backbone scaffold, which may be any appropriate shape, including a sheet, a cylinder, a plurality of cylinders, a prism, a plurality of prisms, a cuboid, a plurality of cuboids, a rectangular cuboid, a plurality of rectangular cuboids, disks, plugs, any combination thereof and the like. The size of the backbone scaffold may be designed according to the required size of the collagen membrane including the backbone scaffold, which may be any appropriate size, depending on the final use thereof. According to some embodiments, the size of the implantable structure of the invention is in the range of 0.5-50 cm². According to some embodiments, the size of the implantable structure of the invention is in the range of 0.5-1.0 cm². According to some embodiments, the size of the implantable structure of the invention is in the range of 1.0-5.0 cm². According to some embodiments, the size of the implantable structure of the invention is in the range of 5.0-20 cm². According to some embodiments, the size of the implantable structure of the invention e is in the range of 20-50 cm². According to some embodiments, the size of the implantable structure of the invention may be altered after preparation, e.g., by cutting or trimming. According to some embodiments, if the size of the implantable structure of the invention is altered after it is prepared, the implantable structure of the invention may undergo an additional process for covering any exposed ends of the backbone scaffold by collagen, as detailed herein.

The invention further provides a method of preparing an implantable structure comprising at least one biocompatible backbone scaffold and at least one biocompatible collagen membranes deposited on at least a part of said backbone surface, said method comprises: providing at least one biocompatible backbone; immersing at least a part of said biocompatible backbone scaffold in a solution comprising collagen; fibrillating said collagen; and crosslinking said collagen thereby providing crosslinked collagen membrane deposited on the surface of said backbone.

In another embodiment, the fibrillated collagen is optionally compressed by applying pressure (e.g. 1 kg) onto said collagen, thereby forming a collagen with high density. In another embodiment, the collagen is compressed before or after the crosslinking step.

The invention further provides a method of preparing an implantable structure comprising at least one biocompatible backbone scaffold and at least one biocompatible collagen membrane, wherein the collagen membrane comprises different crosslinked levels/regions of said membrane deposited on at least a part of said backbone surface, thereby, the degradation rate of said membrane is controllable, the method comprises:
a) providing at least one biocompatible backbone; immersing at least a part of said biocompatible backbone scaffold in a solution comprising collagen; fibrillating said collagen; and crosslinking said collagen thereby providing crosslinked collagen membrane deposited on the surface of said backbone; and
b) immersing partially the collagen-deposited membrane in a crosslinking solution (wherein only part of the collagen-deposited membrane is exposed to a crosslinking solution); thereby providing an implantable structure comprising at least one biocompatible backbone scaffold and at least one biocompatible collagen membrane, wherein the collagen membrane comprises different crosslinked levels/regions of said membrane deposited on at least a part of said backbone surface, thereby, the degradation rate of said membrane is controllable

It is noted that throughout, unless specifically mentioned otherwise, said at least one collagen membrane is deposited on at least a part of the surface of the backbone scaffold. In some embodiments said at least one collagen membrane is deposited on all the surface of said backbone scaffold. When referring to the surface of said backbone scaffold it should be understood to include collagen membranes that coat the outer surface of said backbone but may also include coating of inner surfaces of the backbone and penetrate the backbone scaffold and/or any perforations that the backbone scaffold includes. For example, if the backbone scaffold is in the form of a mesh, the prepared collagen membrane coats, in some embodiments, the mesh on all sides and is further formed in the holes of the mesh, such that the collagen membrane reaches from side to side of the mesh, through those holes.

According to some embodiments, the shape of the collagen membrane is defined according to the shape of the backbone scaffold, accordingly, the backbone scaffold obtained and utilized according to the method above has a shape defined according to the required shape of the prepared collagen membrane.

According to some embodiments, the collagen solution includes at least one fibrillation agent. The collagen in the solution may be fibrillated and crosslinked by any method known in the art, wherein the presence of the backbone scaffold in the solution during the fibrillation and the crosslinking causes the collagen membrane to coat the backbone scaffold.

According to some embodiments, the collagen is fibrillated by neutralizing its pH, e.g., by means of a buffer solution having a neutral or basic pH. According to some embodiments, the fibrillation agents may include one or more bases and/or salts, such as sodium phosphate, Tris HCl, potassium hydroxide or sodium hydroxide.

Once the collagen is fibrillated, it may be crosslinked according to any known method in the art. According to some embodiments, the collagen is crosslinked by at least one of enzymatic mediated process, by a physical treatment (e.g., heat, UV radiation), or by means of a chemical cross-linking agent, wherein the crosslinking agent comprises a reducing agents, such as a reducing sugar or a reducing sugar derivative, or any combination thereof.

According to some embodiments, at least one crosslinking agent comprises an aldehyde or ketone mono sugar or mono sugar derivative wherein the α-carbon is in an aldehyde or ketone state in an aqueous solution. According to some embodiments, at least one crosslinking agent includes compounds and reagents, as detailed in U.S. Pat. No. 6,346,515, which is incorporated herein by reference. As detailed therein, the reducing sugars may form Schiff bases with the α or ε amino groups of the amino acids of the collagen molecule. The Schiff base may then undergo an Amadori Rearrangement to form a ketoamine product. Two adjacent ketoamine groups may then condense to form a stable intermolecular or intramolecular crosslink.

The at least one reducing agent is selected from, for example, glycerose, threose, erythrose, lyxose, xylose, arabinose, ribose, allose, altrose, glucose, mannose, gulose, idose, galactose, talose, or any other diose, triose, tetrose, pentose, hexose, septose, octose, nanose or decose, or any combination thereof. For example, when the crosslinking agent comprises a ribose, a stable crosslink via a pentosidine group may be formed.

As detailed above, the implantable structure of the invention includes at least one collagen membrane and a backbone scaffold, wherein, after a certain period of time, the collagen membrane biodegrades, leaving the backbone scaffold in place to provide support and the like, at a time when infections in the vicinity of the backbone scaffold are less probable. According to some embodiments, the degradation rate of the collagen membrane may be controlled by the extent of the crosslinking between the collagen fibrils. The extent of the cross linking may be controlled, e.g., by the concentration of at least one crosslinking agent, the temperature and the time during which the collagen fibrils are exposed to at least one crosslinking agent. According to some embodiments, the cross-linking may be performed at a concentration of at least one cross linking agent in the range of about 0.01%-5%. According to some embodiments, the cross-linking may be performed at a temperature range of about 20-40° C. According to some embodiments, the cross-linking may be performed for a duration range of about 6-360 hours.

According to some embodiments, the collagen fibrils are contacted with at least one crosslinking agent by introducing at least one crosslinking agent into the collagen solution. According to some embodiments, the collagen fibrils may be placed in a receptacle that allows the exposure of only portions thereof to at least one crosslinking agent. For example, at least one crosslinking agent may be added only to the parts of the receptacle, allowing exposure of only the required portions of the collagen fibrils to at least one crosslinking agent. According to other embodiments, the collagen fibrils are contacted with at least one crosslinking agent by dipping the collagen fibrils formed around the internal scaffold into a crosslinking solution, such that only part of or all of the collagen fibrils may be contacted with at least one crosslinking agent. According to some embodiments, the collagen membrane is prepared such that the parts thereof intended to be implanted in the direction of bone tissue degrade at a higher rate than those intended to be in a direction away from the bone tissue, when it is desirable that the collagen be replaced by bone tissue.

In some embodiments, said implantable structure of the invention comprises at least two collagen membranes. In some embodiments, said at least two collagen membranes are the same. In other embodiments, said at least two collagen membranes are different (for example having different densities, such that the inner membrane closest to the backbone has lower/higher density than the outer membrane, further away from the backbone scaffold, having higher/lower density accordingly).

In some embodiments, the implantable structure of the invention comprises at least one collagen membrane having different densities. In some embodiment, the density of the collagen membrane is higher in the inner membrane (closer to the backbone) and lower in the outer membrane, further away from the backbone). In some embodiment, the density of the collagen membrane is lower in the inner membrane (closer to the backbone) and higher in the outer membrane. The different densities is due to the process for the preparation of the implantable structure which includes a “compressing step”, which yields different density regions of the collagen membrane. In some embodiments, the “compressing step” (e.g. 1 Kg) is done before or after the crosslinking step according to the methods of this invention. In some embodiments, the different density regions/levels of the collagen membrane results in a gradient of degradation rates.

According to some embodiments, the collagen membrane is prepared such that various regions thereof are degraded at different degradation rates. For example, certain regions, which are designed to degrade at a slower rate, are brought into contact with at least one crosslinking agent before the other regions of the membrane, for a certain period of time, after which the entire membrane is brought into contact with at least one crosslinking agent. Accordingly, the regions that are in contact with the at least one crosslinking agent for longer periods of time have a higher degree of crosslinking and accordingly, degrade at a slower rate. In some embodiments, the different degree of crosslinking of the collagen membrane results in gradient of degradation rates.

According to some embodiments, the collagen membrane of this invention comprises regions having different degree of crosslinking, different densities or both. In other embodiment, the degradation rate of the collagen membrane is controllable, based on the degree of the crosslinking of the collagen and/or its density.

According to some embodiments, once the collagen is crosslinked, the prepared collagen membrane comprising the backbone scaffold is washed to remove residues of reactants, such as fibrillation agents, crosslinkers, non-cross-linked-collagen and the like. According to further embodiments, the collagen membrane is then dehydrated by any appropriate means, such as compression, air drying, freeze-drying, critical point drying, or any combination thereof. The drying procedure is devised such that the collagen membrane maintains its three-dimensional shape and such that the procedure does not affect the capability of the collagen component in the collagen membranes to biodegrade. The drying procedure may further sterilize the collagen membranes and render them dry, effectively prolonging their shelf life.

According to some embodiments, the collagen membrane may comprise at least one pharmaceutical active agent having various therapeutic effects. According to some embodiments, at least one pharmaceutical active agent is immobilized within the collagen membrane by at least one crosslinking agent, e.g., the reducing sugars, or by its natural tendency for binding to collagen. During the gradual biodegradation of the collagen membrane, such at least one pharmaceutical active agent is gradually released into the body. Such at least one pharmaceutical active agent may include antimicrobial agents, anti-inflammatory agents, growth factors having tissue regeneration induction properties and the like, as well as any combination thereof.

Antimicrobial agents may include penicillin, cefalosporins, tetracyclines, streptomycin, gentamicin, sulfonamides, and miconazole. The anti-inflammatory agents may include cortisone, synthetic derivatives thereof, and the like. Tissue regeneration induction factors may include differentiation factors, bone morphogenetic proteins, attachment factor and growth factors, such as, fibroblast growth factors, platelet derived growth factors, transforming growth factors, cementum growth factors, insulin-like growth factors, and the like.

According to some embodiments, the implantable structure of the invention may be used in conjunction with a space-maintaining material (“space maintainer”). The term “in conjunction” is intended to cover uses in which the space maintainer is adjacent to the collagen membrane, is attached to the collagen membrane by any appropriate means, or is incorporated into the collagen component of the collagen membrane.

A space maintainer may be used in some procedures in order to maintain a space in which the regenerating cells can migrate and repopulate. In some instances, such a space occurs naturally, for example, when a tumor is excised from a bone. In other instances, such a space is not available, for example, in various types of periodontal or bone lesions. In such instances it may be necessary to insert filling material between the collagen membrane and the regenerating tissues. Examples of space maintainers are (i) hyaluronan (hyaluronic acid), (ii) mineralized freeze dried bone, (iii) deproteinazed bone, (iv) synthetic hydroxyapatite, (v) crystalline materials other than those mentioned under (ii)-(iv), enriched with osteocalcin or vitronectin, and (vi) heat-treated demineralized bone, wherein the bone-derived substances may be of human origin. Also possible are combinations of any of the above space maintainers, such as the combination of hyaluronan and with one or more of the other space maintainers.

For various applications depending on the size, form and location of the regenerating site, the space maintainers may be enriched with one or more of the antibacterial, anti-inflammatory and tissue-inductive factors mentioned above; and/or enriched with a substance intended to aid in maintaining the shape of the space maintainer matrix, e.g. one or more matrix proteins selected from the group comprising collagen, fibrin, fibronectin, osteonectin, osteopontin, tenascin, thrombospondin; and/or glycoseaminoglycans including heparin sulfate, dermatan sulfates, chondrointin sulfates, keratan sulfates, and the like.

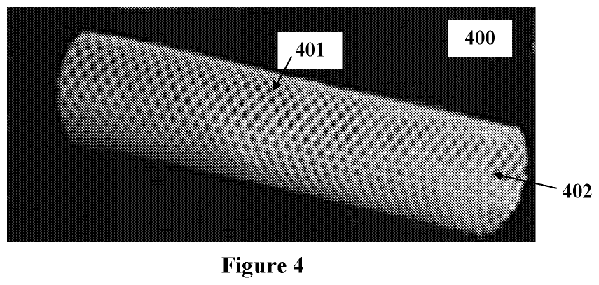

According to some embodiments, the implantable structure of the invention is designed such that it fills the space in which the tissue is to be regenerated. Accordingly, the use of space maintainer may not be necessary, since the tissue may be regenerated and replace the biodegrading collagen component. According to some embodiments, as detailed herein, the implantable structure of the invention may be prepared to have any size or shape, particularly defined according to the size and shape of the internal scaffold. In order for the implantable structure of the invention to fill a certain volume, it may be prepared from a backbone scaffold designed to occupy a predefined volume. For example, the backbone scaffold may be prepared as a three-dimensional entity, having any shape and size, wherein the collagen membrane covers all sides and inner volumes of the internal scaffold. For example, the backbone scaffold may be prepared from a mesh formed into the shape of several adjacent cylinders, spheres, prisms, or any combination thereof or any wavy or three dimensional or partially three-dimensional shape. According to some embodiments, the collagen membrane may coat the backbone scaffold and may or may not fill any or all of the inner volume and/or voids of the backbone scaffold, e.g., the inner volume of a mesh cylinder. When implanted, any surrounding tissue may, over time, replace the biodegrading collagen membrane, including in any inner volumes formed by the backbone scaffold. An example of a three-dimensional backbone scaffold, comprising an inner volume is presented in FIG. 4.

The invention is further directed to a kit comprising an implantable structure comprising backbone scaffold and at least one collagen membrane. The invention further provides a kit comprising at least one biocompatible backbone scaffold, at least one solution of collagen, and instructions for use thereof. According to some embodiments, the kit further comprises at least one of a crosslinker, a fibrillation agent or both. Further embodiments are directed to the use of the implantable structure as an implanted device, e.g., a dental or orthopedic bone regeneration implanted device, a hernia repair implanted device, a dura repair implanted device. For example, the collagen membrane may be used for repairing skull fractures as well as nonunion fractures.

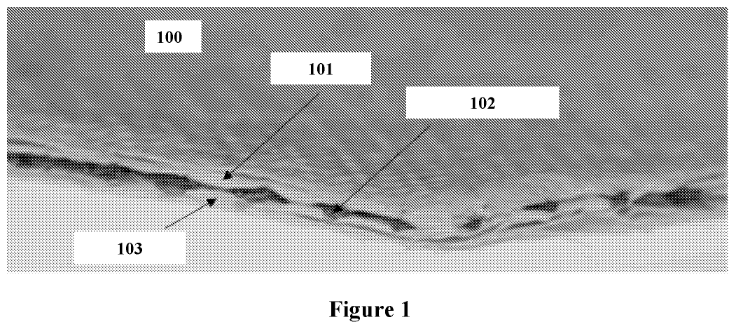

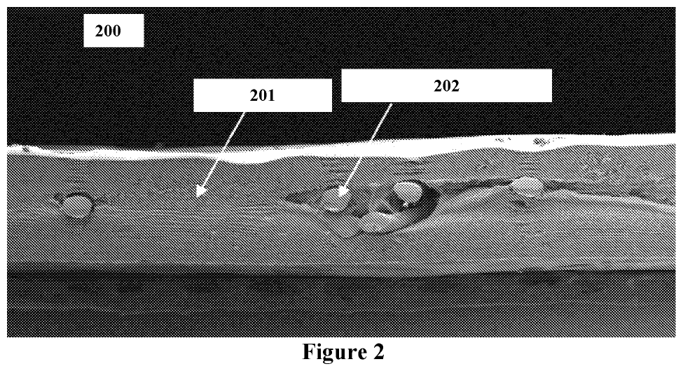

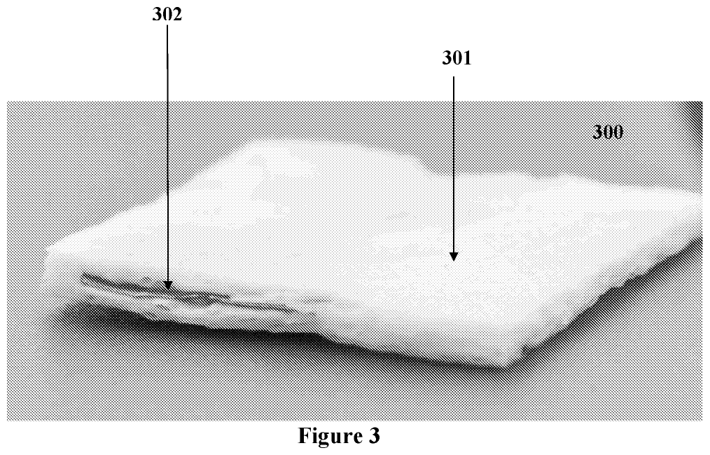

Reference is made to FIGS. 1-3, presenting embodiments of implantable structures according to the invention. FIG. 1 shows an implantable structure (100), wherein collagen membranes (101 and 103) cover the outer surface of an internal backbone scaffold is a metal mesh (102) and further intertwined therethrough. FIG. 2 shows an implantable structure (200) having a polymeric mesh (202) as a backbone scaffold and a collagen membrane (201) that covers the mesh and is further intertwined therethrough. In FIG. 3, an implantable structure of the invention (300) is formed of a backbone scaffold of titanium mesh (302) and a collagen membrane (301) that covers the mesh and is further intertwined therethrough. FIG. 4 presents an example of a three-dimensional implantable structure of the invention (400) having an outer surface (401) and an inner void (402), although not shown the backbone scaffold mesh forming the three-dimensional structure of the implant of the invention is covered with a collagen membrane and is further intertwined therethrough.

In order to better understand how the present invention may be carried out, the following examples are provided, demonstrating a process according to the present disclosure.

### EXAMPLES

### Example 1-Preparation of Collagen Membrane on Biocompatible Backbones Having Different Densities

An aliquot of 640 ml of collagen was mixed with 60 ml of fibrillation buffer composed of 200 mM sodium phosphate having a pH 11.2. After a short mixing the collagen was poured into a molding plate of 11×16 cm. A stainless-steel mesh was submerged up to about 1.7 cm from the bottom. Fibrillation was allowed to proceed for 18 hours at 37° C. The resulted gel was compressed using 1 Kg weight for 20 hours at 37° C. The collagen/stainless steel mesh was crosslinked in a medium of 1% ribose, 70% ethanol and 29% PBS for 11 days at 37° C. The prepared collagen membrane was then washed with water and dried by lyophilization. FIG. 1 presents the prepared collagen membrane.

The same procedure was followed using a polymeric mesh instead of the stainless-steel mesh, providing the collagen membrane presented in FIG. 2. Further, the same procedure was followed using a titanium mesh, providing the collagen membrane presented in FIG. 3.

### Example 2—Preparation of Collagen Membrane Having Different Degree of Crosslinking and Different Densities on Biocompatible Backbones

In order to prepare a collagen membrane having a slow rate degradation region and a high rate degradation region, an aliquot of 640 ml of collagen is mixed with 60 ml of fibrillation buffer composed of 200 mM sodium phosphate having a pH 11.2. After a short mixing the collagen is poured into a molding plate. A stainless-steel mesh is immersed in the solution. Fibrillation is allowed to proceed for 18 hours at 37° C. The resulted gel is compressed using 1 Kg weight for 20 hours at 37° C. The collagen/stainless steel mesh is crosslinked in a medium of 1% ribose, 70% ethanol and 29% PBS.

In order to form an uneven collagen membrane on the mesh, at first the entire collagen/stainless steel mesh is immersed in the crosslinking medium for a period of about 5-10 days at 37° C. After a predefined time period, the forming membrane is partially removed from the crosslinking medium, e.g., by suspending the forming membrane on the surface of the medium, such that one face thereof is in/on the medium and the other face thereof is exposed to the surrounding atmosphere. The mesh is held in place for about 5-10 days by any appropriate means, such that the collagen continues to be crosslinked on the side or part of the mesh that is in contact with the medium, though not on the side or part of the mesh that is not in contact with the medium. Thus, a collagen component is formed unevenly on the mesh, wherein the crosslinking degree of one side thereof is higher than that of the other side. Accordingly, two regions are formed—one having a high degree of crosslinking and therefore, a slow degradation rate and the other having a relatively low crosslinking degree and therefore, a high degradation rate.

The formed collagen membrane is then washed with water and dried by lyophilization. The same procedure is followed using a polymeric mesh or titanium instead of the stainless-steel mesh.

While certain features of the invention have been illustrated and described herein, many modifications, substitutions, changes, and equivalents may occur to those skilled in the art. It is, therefore, to be understood that the appended claims are intended to cover all such modifications and changes as fall within the true spirit of the invention.

1. An implantable structure comprising at least one biocompatible backbone scaffold and at least one biocompatible and biodegradable collagen membrane deposited on at least a part of said backbone surface; wherein the biodegradation rate of the membrane is controllable.

2. The implantable structure according to claim 1, wherein said backbone scaffold is formed of at least one of titanium, nitinol, polytetrafluoroethylene (PTFE, Teflon®), stainless steel, polypropylene, polystyrene, polyester, silicon, or any combination thereof.

3. The implantable structure according to claim 1, wherein said backbone scaffold is in the form of a woven or non-woven mesh, wires, rods, or any combination thereof.

4. The implantable structure according to claim 1, wherein the collagen membrane further comprises at least one pharmaceutically active agent.

5. The implantable structure according to claim 4, wherein said pharmaceutically active agent is selected from antimicrobial agents, anti-inflammatory agents, factors having tissue regeneration induction properties and any combination thereof.

6. The implantable structure according to claim 1, wherein the collagen membrane comprises crosslinked collagen.

7. The implantable structure according to claim 1, further comprising a space maintainer.

8. A method of preparing an implantable structure as defined in claims 1 to 7, said method comprising: providing at least one biocompatible backbone; immersing at least a part of said biocompatible backbone scaffold in a solution comprising collagen; fibrillating said collagen; and crosslinking said collagen thereby providing crosslinked collagen membrane deposited on the surface of said backbone.

9. The method according to claim 8, wherein the solution comprises at least one fibrillation agent.

10. The method according to claim 9, wherein said at least one fibrillation agent is selected from sodium phosphate, Tris HCl, potassium hydroxide or sodium hydroxide.

11. The method according to claim 8, wherein the collagen is crosslinked by at least one of an enzymatic mediated process, heat, UV radiation, at least one crosslinking agent or any combination thereof.

12. The method according to claim 11, wherein said at least one crosslinking agent is selected from glycerose, threose, erythrose, lyxose, xylose, arabinose, ribose, allose, altrose, glucose, mannose, gulose, idose, galactose, talose, or any other diose, triose, tetrose, pentose, hexose, septose, octose, nanose or decose, or any combination thereof.

13. The method according to claim 8, wherein the collagen comprises one or more regions, wherein each region is crosslinked to a different degree of crosslinking.

14. The method according to claim 8, further comprising washing the collagen membrane to remove residual reactants.

15. The method according to claim 8, further comprising dehydrating the collagen membrane.

16. The implantable structure according to any one of claims 1 to 7, for use in at least one of dental or orthopedic bone regeneration, hernia repair, dura repair and any combinations thereof.

17. The implantable structure according to nay one of claims 1 to 7, for use in conjunction with at least one space-maintainer.

18. A kit comprising an implantable structure according to any one of claims 1 to 7.

19. A kit comprising at least one biocompatible backbone scaffold, at least one solution of collagen, and instructions for use thereof.

20. The kit according to claim 19, wherein said kit further comprises at least one fibrillation agent.

21. The kit according to claim 19, wherein said kit further comprises at least one crosslinker.
